(19) United States
(12) Patent Application Publication
(10) Pub. No.: US 2016/0131626 A1
(43) Pub. Date: May 12, 2016

(54) METHODS FOR TRACING FLUID MIGRATION

(71) Applicant: New Century Financial Capital, LLC, The Woodlands, TX (US)

(72) Inventor: Jaime Cuevas Dermody, Delray Beach, FL (US)

(73) Assignee: New Century Financial Capital, LLC

(21) Appl. No.: 14/939,239

(22) Filed: Nov. 12, 2015

Related U.S. Application Data
(60) Provisional application No. 62/078,642, filed on Nov. 12, 2014.

(51) Int. Cl. G01N 33/00

(52) U.S. Cl. CPC G01N 33/0036

(57) ABSTRACT

The disclosure relates to application of a signature elemental tracer referred to herein as a signature). It comprises a selection of a small amount of a unique combination of a non-radioactive isotope or variant of a particular element or elements with stable isotopes, different from the natural-occurring combination of the elements in question. In particular, there are 54 such stable multi-isotopic elements and 17 rare-earth elements available for the creation of signature. The signature can be chosen based on samples of traced material and the environment it is to be used in, over time based on detectability, duration, and robustness in that environment. By utilizing isotope combinations as a signature, one can readily determine with a mass spectrometer, whether the traced material in question was or was not in a particular sample of contamination, plant tissue, animal tissue, or associated with some event of interest.

### CROSS-REFERENCE TO RELATED APPLICATIONS

This application claims priority to U.S. Provisional Application No. 62/078,642, filed on Nov. 12, 2014, entitled “Methods for Tracing Fluid Migration,” the contents of which are hereby incorporated by reference in their entirety.

### BACKGROUND

Tracer materials have a large and expanding use, serving in many applications across numerous fields. In particular, tracers measure occurrences in many technical and industrial activities. Novel tracer materials are thus of major practical interest. Industrial fluid tracers were first introduced to measure and model fluid flow in vessels. Such models are used for fluid or gas in chemical reactors and other process units, rivers, and the ocean, as well as through soils and porous structures. In medicine, they are can be used to study the flow of chemicals, harmful or not, in the blood streams of animals and man. In various industrial processes, tracers can be used to identify the source of any fluid and gas transported from one place to another (i.e., distribution), even over many years. Tracers are often commonly used for determining the location and severity of leaks.

In oil and gas operations, many different fluids are introduced, produced, or moved during exploration, production, transportation, retailing, and use. These fluids include: those manufactured to perform specific functions (e.g., drilling mud), those collected from different formations in a well, and refined liquid and gas products. Some of these fluids collected were injected into the well or reservoir rocks, returned to the surface during operations (e.g., drilling muds and fracking fluids). Other fluids occur naturally in the formation rocks, for example, oil, gas condensate, and water. Some fluids collected are a mixture of those naturally occurring and artificially introduced.

During the life of an oil or gas field, many fluids, solids (including granular materials), and gases are put into the ground, and much of that is returned during production. Other fluids and gases are used in processing, transportation, and refining. It is important to understand how such fluids and gases move and change during each of those stages, in reservoir rocks and soil, wells, pipelines, holding ponds, tanks, and equipment. The information gleaned from this understanding can inform the efficiency of those stages. In particular, it shows: how certain specialized fluids and gases are working (e.g., stimulation fluids), whether fluids and gases are moving from one production zone to another (e.g., through the reservoir rock or behind seals in a well that are meant to isolate zones), leakage, and fluids or gases migration into aquifers.

There are growing concerns that some fracking operations in shale formations create paths through which artificial or naturally-occurring fluids and gases flow into aquifers, which did not exist before fracking Early identification of the presence or absence of such flows would be of great value to the operator, regulators, and society. In addition, a better understanding of these fluid and gas flows allow the operator to avoid environmental dangers and/or improve productivity and safety. Similar concerns and opportunities exist in various manufacturing and agricultural operations.

One practice used to understand fluid flow is called a tracer. The tracer is added to a fluid at one point in the process and detected later. There are tracer technologies in the oil and gas industry of two types:
(1.) Nonreactive easily-differentiated material (dyes, paints, glitter), that are blended into the drilling mud so as to be identified when the fluid returns to surface and reveal the circulation time; and
(2.) Chemical markers that are added to fluids in the continuous phase of drilling, coring, or completion that are used to identify the filtrate in cores or fluids samples, as well as, to identify refined products.
   Such tracers, in oil and gas fields, become part of the filtrate, remaining in solution and moving with the filtrate through permeable zones. They are intended to: not be absorbed in clays or degrade at a predetermined acceptable rate be measureable in trace amounts, and be safe to handle. Examples include: weakly-emitting radioisotopes (e.g., bromide or iodide compounds that do not occur naturally in most muds or reservoirs), and nitrate anion added as sodium-, potassium-, or calcium-nitrate (one of the earliest tracers, which degrades and is difficult to detect and analyze).

These methods provide a single tracer that cannot be distinguished among multiple fluids and/or adjacent wells, and cannot identify long-term events due to degradation. As a result, mathematical modeling from core samples and ground-penetrating radar is still the primary method of estimating fluids flows within, between, and beyond reservoirs.

A tracer is needed that is: (1.) detectable in very small amounts; (2.) readily identifiable with mass spectroscopy; (3.) robust for hundreds of years in extreme well environments and across adjacent geological and geographical circumstances; (4.) not dangerous to handle or to the environment; and (5.) unique to each well, field, or batch.

### SUMMARY

In at least one aspect, the disclosure relates to application of a signature-elemental tracer, consisting of a selection of one or more stable non-radioactive isotope of a single-isotope or multiple-stable-isotope solid, liquid, or gaseous elements. A selection used for the present disclosure would be different than elements occurring naturally in the region of the drill, production, or use site. In an embodiment, about one kilogram is dissolved in a million liters of fluid or gas. Example of such elements are rutheniums, which has seven stable isotopes (atomic mass numbers 96, 98, 99, 100, 101, 102, and 104; tin, which has ten stable isotopes atomic mass numbers 112, 114, 115, 116, 117, 118, 119, 120, 122, and 124; xenon, which has nine stable isotopes atomic mass numbers 124, 126, 128, 129, 130, 131, 132, 134, and 136; mercury, which has seven stable isotopes atomic mass numbers 196, 198, 199, 200, 201, 202 and 204; and rare earth elements and/or carbon 13 and 14 variations not found in the region in question, or in combinations not found there.

The particular element or set of elements chosen, for the signature tracer to be used in any given physical location, is one that is readily dissolvable in the solution to be traced; has been optimized in laboratory tests of the (solid, fluid, and/or gas) environments and conditions across which the signature is to be traced, e.g., across a succession of soil, ground water, and rocks areas near a drilling location, and through pipe lines and/or tanks, refineries, retail locations, and consumer equipment or locations; irrigation water and other inputs used in an agricultural grove or farm that will be absorbed by the trees, animals, and/or produce, move through processing facilities, commercial products, transportation modes, distribution facilities, and in consumers themselves; and inputs in a bottling plant that will move through the plant. This includes liquids or gases that will transform into a different state of gas, liquid, or solid, e.g., liquids and gasses that will become solids, like vinyl or plastics. We will refer to the liquid, gas, or solids to be traced a “traced material”. The test metric is to be a non-natural combination of an element or elements, that for hundreds of years is very likely to remain distinct, readily identifiable with an ordinary mass spectrometer, and mobile with the traced material. There are 54 stable multi-isotopic elements. On testing a sample, one can readily prove or disprove that a particular fluid, solid, or gas with such tracer was linked to the source of a particular contamination, injury, event, or condition of interest.

As disclosed herein, a method of tracing can include performing baseline characteristic analysis of a traced material movement areas. The method can include obtaining samples of all the environments that the tracers will experience (e.g., from drill or mining cuttings and geological studies, and from data about processing systems, and from tissues or air samples). The method can include selection in lab test simulations of environment (including the various physical and chemical environments) and a signature element and variety or combination of multiple-stable isotope tracer of an element or elements dissolved in the traced material, among the myriad possible candidates element or sets of elements, that is best in the combination of: readily and persistently dissolved in such environments, easily-identifiable by a simple mass spectrometer, for hundreds of years in such environments, and distinct from other tracers in the same or other traced material that are used in relevant proximity to other tracers/traced material combinations, that they have a reasonable probability of appearing together with in a future sample. The method can include producing and delivering, to the site where the tracing will start, a kilogram of the selected tracer for easy introduction and dissolving in the traced material. The method can include dissolving such tracer in the traced material, that is to be traced through the environments in question and insuring the integrity of such dissolving. The method can include establishment and refinement of methods to readily identifying the tracer selected with a simple mass spectrometer, e.g., in the tissue of people, plants, or livestock, and in the water, air, or commercial products. The method can include taking samples of the environment (e.g., ground water, returning drilling fluids and muds, tissues, partially refined or processed material, and the relevant fluids gases or solids in the various states of use) to monitor the material being traced; and taking relevant samples and determining whether the tracer is extant in such sample to determine if the tracer is linked to any event in question.

In an embodiment, a method of tracing gas, fluid, or solid movement is disclosed. The method can include providing a signature elemental tracer in an amount of traced material, the signature elemental tracer being a single or multiple isotope combination of an element or elements selected from the periodic elements (including rare-earth elements). Selection is based on tests of the fluid to be traced and a field environment the signature elemental tracer is to be used in, under laboratory conditions that mimic the chemical and physics of the field environment. The method can include, after movement of the traced material in the field environment, obtaining a sample of the traced material. The method can include analyzing the sample for the presence of the signature elemental tracer.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF FIGURES

FIG. 1 shows a schematic of an example fracking operation.

FIG. 2 shows a table of stable non-radioactive isotopes of a single-isotope or multiple-stable-isotope solid, liquid, or gaseous elements, which may be used as a signature tracer in accordance with embodiments of the present disclosure.

FIG. 3 shows a flowchart for a method in accordance with embodiments of the present disclosure.

FIG. 4 shows a flowchart for a method in accordance with embodiments of the present disclosure.

### DETAILED DESCRIPTION

In the following description, numerous details are set forth to provide an understanding of the present disclosure. However, it will be understood by those skilled in the art that the present disclosure may be practiced without these details and that numerous variations or modifications from the described embodiments are possible.

The present disclosure relates to methods for tracing fluid, solid, or gas (“traced material”) with a signature tracer, such as in a well system, fracking operation, aquifer, agriculture operation, transportation system, refining, manufacturing, production, and/or distribution system; involving the traced material. Fluids, gases and solids, as referred to herein, includes those materials that will be used to create other fluids, gasses, and/or solids. The method is directed to an inexpensive, safe procedure of placing about one kilogram of a selection of one or more stable (i.e., non-radioactive) isotopes of a particular single- or multiple-isotope element or elements in a traced material that does not occur naturally in the trace environment. We call this the “signature” for a particular traced material in a particular trace environment. There are 54 currently-known stable multi-isotopic elements and 17 rare-earth elements. Additional stable multi-isotopic elementals could be discovered and used as envisioned here.

For instance, silver (Ag) occurs universally in nature in this unique combination of stable isotopes: 51.839% 107-molecular-weight and 48.161% 109-molecular-weight. If silver tested best in a particular sample environment, then about one kilogram of a 25%/75% combination of those isotopes will quickly dissolve in a few million liters of a fluid (that may be used to produce a solid, gas, and/or other fluid) and remain a detectable oddity to any chemist with a standard mass spectrometer for hundreds of years. This would be due to the stability of Ag in that traced material and environment, as lab and field tested. In practice, subsequent tracers in traced material in relevant proximity to previous tracing, would employ a different signature, i.e., a different combination of the same or different element or elements to ensure the individuality of the signatures as needed.

Furthermore, preliminary baseline characteristic analysis of the site environment and traced material in the lab and field allows for selection of the most efficacious signature. Features that may be considered for each signature in a given environment for a given fluid can include, relative mobility, persistence, and ease of detectability in various potential environments over hundreds of years.

These selection of signatures for a particular traced material, or set of potential traced materials, will often be performed in a reaction vessel or column filled with materials from the environment it is to be traced in (e.g., from the cuttings or process environment) under physical and chemical conditions that mimic the environment over hundreds of years. The selection will often also include field tests in the environments that the traced material is to be traced in.

Signatures introduced into the ground will sometimes form mixtures with the naturally occurring form of that element. Detection of the signature will be dependent on how different the signature is from that of the natural form of the elemental in the signature and the environment. Additional considerations in selecting a particular signature for use at a site include adjacent environments.

Matching the signature sampled in delivered traced material, or from spill samples, is indicative that the delivered or spilled material is from the shipped material that contained the signature. This identification is desirable in circumstances of spoilage, contamination, and where authentication is important. The signature of the present disclosure has particular applicability in the shipment of crude oil, refined oil and bulk products, grains, agriculture produce, livestock, processed and unprocessed chemicals, manufactured products, and packaging. In particular, the signature tracer of the present disclosure may be employed in manufacture, shipment, and deployment, of a pollutant, hazardous material, or a toxic material. As such, the invention has particular applicability in the identification of spilled shipments, pesticides, poisonous and toxic compounds, flame retardants, explosives, military chemical and biological agents, naphthalene, and biphenols.

The number of uniquely-identifiable combinations of variants or single- and multi-isotopic stable elements makes the chance that material other than traced material will contain the same signature very small.

Detection of the signature in a sample of traced material may be readily achieved by a multitude of existing and future methods, including: fluorescence and mass spectroscopy, nuclear magnetic resonance spectroscopy, thin-layer and gas chromatography, internally-coupled plasma (ICP-MS), high-performance liquid chromography (HPLC), and ultraviolet visible (UV-VIS). Since the stable multi-isotopic signatures disclosed here do not occur naturally in the combinations envisioned to be used, the signature will be readily detectable as an oddity in any of these methods.

In addition to traced-material tracking, one of the potential uses of the signature of present disclosure is expected to be in proving the innocence or the guilt of environmental wrongdoing.

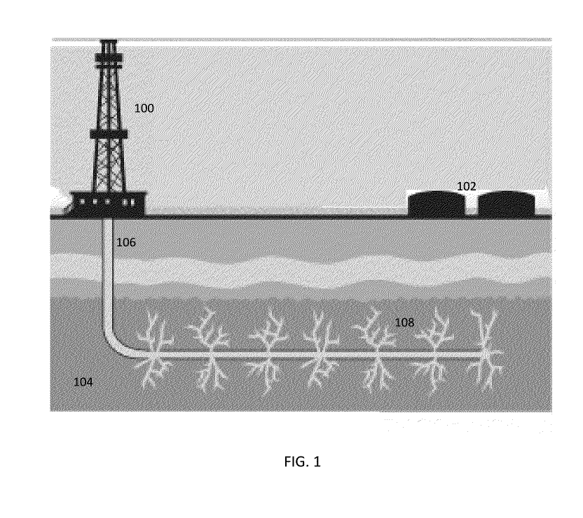

Turning now to FIG. 1, a schematic for an example fracking operation is shown. In a fracking operation, millions of gallons of water, sand, and chemicals are injected through surface drilling equipment 100, at very high pressure into a well 106. The water, sand, and chemicals are usually provided by pumper trucks and storage tanks, and mixed by blenders. The highly pressurized mixture causes the rock layer, such as shale formation 104, to crack in many fissures 108. The fissures are then propped open by the sand particles, such that natural gas from the shale formation 104 can flow up into the well 106. In many fracking operations, the depth of the well 106 may be as deep as 10,000 feet below the surface.

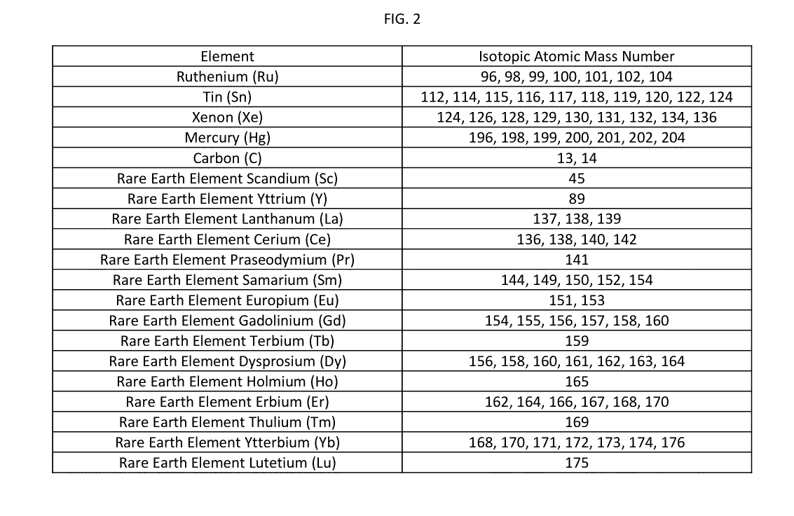

Turning now to FIG. 2, a chart is provided listing examples of stable isotopic elements from which a signature tracer can be selected in accordance with the present disclosure. As explained above, there are 54 currently-known stable multi-isotopic elements and 17 rare-earth elements. These include, Ruthenium, Tin, Xenon, Mercury, Carbon, Scandium, Yttrium, Lanthanum, Cerium, Praseodymium, Smarium, Europium, Gadolinium, Terbium, Dysprosium, Holmium, Erbium, Thulium, Ytterbium, and Lutetium. The isotopic atomic mass number for each stable isotope is listed in FIG. 2.

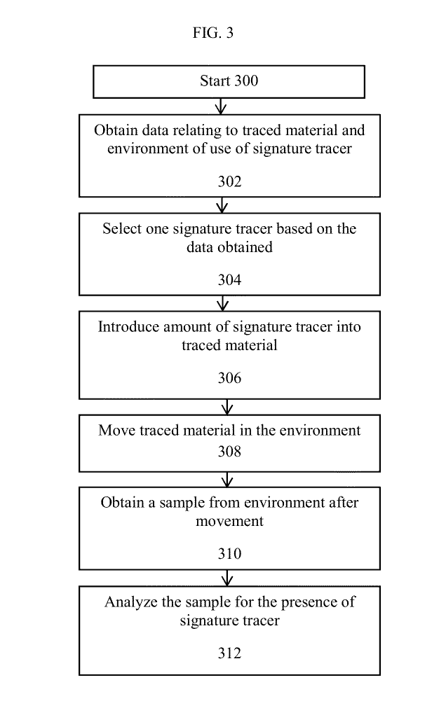

Turning now to FIG. 3, a flowchart is provided for a method in accordance with embodiments of the present disclosure. The method begins at 300. At 302, the method includes obtaining data relating to traced material and the environment in which the signature tracer is to be used. For example, step 302 may include obtaining baseline characteristics such as, for example but not limited to, relative mobility, persistence, and ease of detectability for the particular environment that the material is to be traced in. The data may be obtained by look-up, by simulation, or by actual lab or field testing. Based on the data obtained at 302, the method continues with selecting at 304 one signature tracer from the list of isotopes. At step 306, the method proceeds with introducing an amount of the selected signature tracer into the traced material (such as, for example, pouring the signature tracer into water, sand, and chemicals in a blender for use in a fracking operation).

The traced material is then moved (at 308) in the environment for which the signature tracer was selected for use. In an embodiment, this can mean injecting fracking fluid into a well in a fracking operation. In an embodiment, this can mean injecting fluid in a steam lift operation. In an embodiment, this can mean transporting a fluid via various modes of transportation including a pipeline.

At step 310, the method includes obtaining a sample from the environment after movement of the traced material. This can include, for example, but not limited to, taking ground water samples, taking samples of flow back fluids, sampling after a spill, and the like. The method concludes with analyzing the sample for the presence of the signature tracer. Analysis can be in the form of, for example but not limited to, spectral analysis.

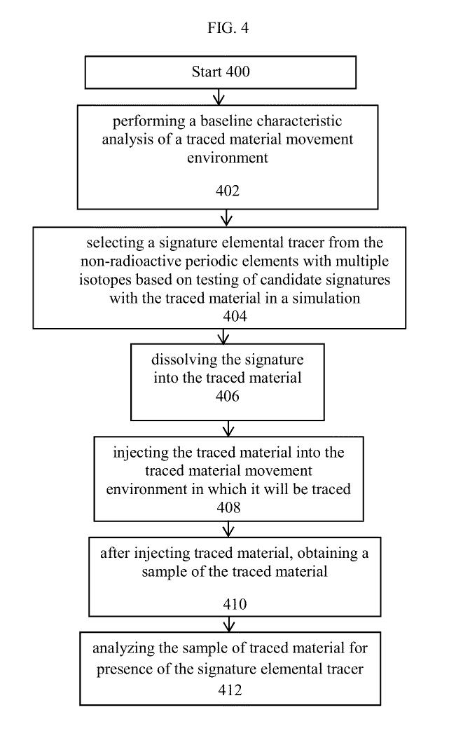

Turning now to FIG. 4, a flowchart is provided for a method in accordance with embodiments of the present disclosure. The method 400 shown is for tracing a traced material's movement in an oil, gas, or mining operation or other industrial, agricultural, logistical, or commercial environment, system, or process. The method includes first, at 402, performing a baseline characteristic analysis of a traced material movement environment. In an embodiment, the baseline characteristic analysis can include a simulation or lab or field testing based on samples or known data for the material to be traced as well as the environment in which the traced material will be used. The baseline characteristic analysis can include evaluation of one or more of the following: composition of rock, soil, or water; composition of traced material; and the environment, material, process, or system, through which the traced material will be traced including one or more of reactivity, absorption, temperature, hydrology, industrial environment, and transitive character.

The method next includes, at 404, selecting a signature elemental tracer from the non-radioactive periodic elements with multiple isotopes based on testing of candidate signatures with the traced material in a simulation of the traced material movement environment that provide similar physical and chemical conditions. The signature tracer can be selected from the elements listed in FIG. 2, for example. The method next includes, at 406, dissolving the signature into the traced material. In an embodiment, the amount of the signature tracer will be minute relative to the overall volume of the traced material in which it is used. In a preferred embodiment, the amount of the signature tracer will be on the order of one kilogram of signature elemental tracer in a few million liters of gas, fluid, or solid gas in the traced material.

The method next includes, at 408, injecting the traced material into the traced material environment in which it will be traced, and allowing time for movement of the traced material. The movement of the traced material can include, for example but not limited to, an enhanced oil, gas, energy, flooding, or other natural-resource recovery operation, such as thermal injection in the form of steam flooding, injection of other fluids, such as surfactants, polymers, or carbon dioxide super fluids, or transportation of the traced material or material associated with the traced material. After injecting traced material, the method includes, at 410, obtaining a sample of the traced material. The method concludes, at 412, with analyzing the sample for presence of the signature elemental tracer.

Although a few example embodiments have been described in detail above, those skilled in the art will readily appreciate that many modifications are possible in the example embodiments without materially departing from this disclosure. Accordingly, such modifications are intended to be included within the scope of this disclosure as defined in the following claims. In the claims, means-plus-function clauses are intended to cover the structures described herein as performing the recited function and not simply structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. §112, paragraph 6 for any limitations of any of the claims herein, except for those in which the claim expressly uses the words ‘means for’ together with an associated function.

The Abstract of the Disclosure is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, the claimed subject matter may be directed to less than all of the features of any of the disclosed embodiments.

What is claimed is:

1. A method of tracing gas, fluid, or solid movement, comprising:
providing a signature elemental tracer in an amount of traced material, the signature elemental tracer being an single or multiple isotope combination of an element or elements selected from the periodic elements;
wherein selection is based on a baseline characteristic analysis of the fluid to be traced and of a field environment the signature elemental tracer is to be used in;
after movement of the traced material in the field environment, obtaining a sample of the traced material; and
analyzing the sample for the presence of the signature elemental tracer.

2. The method according to claim 1, wherein the analyzing comprises mass spectroscopy or other spectral analysis methods.

3. The method according to claim 1, wherein movement of the traced material comprises injection or other introduction in one of a hydraulic fracturing operation, a drilling operation, an industrial operation, a transportation operation, and an agriculture operation.

4. The method according to claim 1, wherein movement of the traced material comprises one or more of recovery, reuse, and disposal of the traced material.

5. The method according to claim 1, wherein movement of the traced material comprises an enhanced oil, gas, energy, flooding, or other natural-resource recovery operation, such as thermal injection in the form of steam flooding, or injection of other fluids, such as surfactants, polymers, or carbon dioxide super fluids.

6. The method according to claim 1, wherein movement of the traced material comprises transportation of the traced material or material associated with the traced material.

7. The method according to claim 1, further comprising: selecting the signature elemental tracer from the available periodic elements, based on at least one characteristic of the field environment in which the traced material movement occurs or is situated.

8. The method according to claim 6, wherein at least one characteristic of the environment in which the traced material movement occurs comprises one or more of the following: composition of rock, soil, or water; composition of traced material; and the environment, material, process, or system, through which the traced material will be traced including one or more of reactivity, absorption, temperature, hydrology, industrial environment, and transitive character.

9. The method according to claim 1, wherein providing a signature in an amount of traced material further comprises dissolving an amount on the order of one kilogram of signature elemental tracer in a few million liters of gas, fluid, or solid gas in the traced material.

10. The method according to claim 1, wherein the signature tracer does not emit gamma rays.

11. A method of tracing the traced material movement in an oil, gas, or mining operation or other industrial, agricultural, logistical, or commercial environment, system, or process, comprising:
performing a baseline characteristic analysis of a traced material movement environment;
selecting a signature elemental tracer from the non-radioactive periodic elements with multiple isotopes based on testing of candidate signatures with the traced material in a simulation of the traced material movement environment that provide similar physical and chemical conditions;
dissolving the signature into the traced material;
injecting the traced material into the traced material movement environment in which it will be traced;
after injecting traced material, obtaining a sample of the traced material; and
analyzing the sample of traced material for presence of the signature elemental tracer.

12. The method according to claim 11, wherein the analyzing comprises mass spectroscopic and other identification.

13. The method according to claim 11, wherein the fluid movement area comprises a hydraulic fracturing, drilling, mining, or geo-thermal operation.

14. The method according to claim 11, wherein the fluid movement area comprises a recovery or traced material and/or flooding operation.

15. The method according to claim 11, wherein the fluid movement area comprises injection of the fluid in an enhanced oil or gas recovery operation or other mining, such as thermal injection in the form of steam flooding, or injection of other fluids, such as surfactants, polymers, or carbon dioxide super fluids.

16. The method according to claim 11, wherein the traced material movement area comprises an area of transportation, processing, distributing, retailing, or other sale of the gas, fluid, or solid traced material.

17. The method according to claim 11, wherein the traced material movement area comprises an industrial, commercial, agricultural, and/or storage fluid vessel.

18. The method according to claim 11, wherein the baseline characteristic analysis comprises evaluation of at least one characteristic of the traced material movement area, the characteristic comprising one or more of the following: composition of rock, soil, fluids, produce, plants, people, or livestock, reactivity, absorption, temperature, hydrology, and/or transitive character.

19. The method according to claim 11, wherein dissolving the signature in the traced material comprises dissolving an amount on the order of kilogram of the signature per few million liters of fluid or gas.

20. The method according to claim 11, wherein the signature does not emit gamma rays.
